(19) United States
(12) Patent Application Publication
(10) Pub. No.: US 2007/0129660 A1
(43) Pub. Date: Jun. 7, 2007

(54) Tissue treatment device

(76) Inventors: Alastair G. McLeod, Rugby (GB); Stephen J. Cook, Reading (GB)

Correspondence Address: DEWITT ROSS & STEVENS S.C., 8000 EXCELSIOR DR, SUITE 401, MADISON, WI, 53717-1914, US

(21) Appl. No.: 10/597,988

(22) PCT Filed: Feb. 23, 2005
(86) PCT No.: PCT/GB05/00679

§ 371 Date: Aug. 15, 2006

(30) Foreign Application Priority Data
Feb. 24, 2004 (GB) 0403959.9

(51) Int. Cl. A61M 1/00

(52) U.S. Cl. 602/41; 604/313

(57) ABSTRACT

The tissue treatment device consists of a bladder (2), having inlet (1) to be connected to a fluid source (not shown). The bladder (2) is placed within a wound cavity and inflated to conform to the cavity shape and apply constant pressure against the wound surface or pulsed pressure, as required. A seal (3) isolates bladder (2) and the wound cavity from atmosphere. Seal (3) includes one or more inlets/outlets (4, 5) for connection to a vacuum source and/or for the supply of media to be introduced into the wound cavity. The vacuum source can be a vacuum pump or a continuous vacuum provided in hospitals. The media to be introduced into the wound cavity can be saline, water or ozone, oxygen, or any substance to promote wound healing. The bladder (2) is made from polymeric material and can also be impregnated with wound healing compounds to promote healing.

This invention relates to a wound healing device, in particular a device using vacuum or suction therapy for enhanced drainage of the wound to promote wound healing.

With the ageing of the world's population the clinical and cost impact of several categories of soft tissue wounds is increasing including pressure ulcers, vascular ulcers and diabetic foot ulcers. Not only is it required to accelerate healing of such wounds compared with conventional methods but also to succeed in healing the minority which are impossible to heal with conventional methods.

A solution to this problem not only reduces suffering by patients but also the considerable cost of conventional wound care products and the associated healthcare labour.

It has been known since the 1980s that the application of a vacuum to a wound by its suction action provides an antiseptic effect, promotes wound clearing from bacteria, pus and necrotic masses and promotes quicker filling of the wound with granulation tissues, thereby providing a faster rate of healing.

Prior art vacuum systems comprise a cover to seal the wound and a vacuum applied to the wound surface. Typically, the vacuum is applied to the wound surface by means of a suction pump with drainage tube within a porous dressing provided under the cover for the wound space so that exudates are siphoned from the wound. Alternatively, the suction pump with drainage tube or tubes is attached to a flexible pouch which is either porous or with holes on its surface so that exudates are drawn from the wound space into the pouch to be carried away from the wound.

The present invention aims to make improvements. Accordingly, the present invention provides a device for treatment of a wound comprising an inflatable bladder to be placed over a wound, the bladder inflated to conform to the wound surface, sealing means to isolate the bladder and the wound surface from the atmosphere and vacuum means to apply a vacuum to the area between the bladder and the wound and drain exudates from the wound. The bladder is inflated to take up the area within the wound cavity that is normally filled with a packing material such as sponge or similar. Such packing material has its problems of retaining wound exudate within it, becoming enmeshed with the tissue within the wound and tearing the tissue ingrained upon removal of the dressing. Also, the bladder is not used to convey the exudates from the wound as with prior art devices and advantageously continues to apply an even pressure against the wound cavity surface which is comfortable even when the body rests against the device.

In a preferred embodiment, the inflated bladder applies an alternating pressure against the wound cavity surface, to induce perfusion of the wound surface for quicker healing. Preferably, the bladder comprises a surface area of greater magnitude to that of a wound cavity within which it is to be used in order to provide folds of the bladder against the wound cavity surface when inflated. The folds provide areas between the bladder and the wound surface for suction. Preferably, the bladder comprises convolutions within its outer surface or more preferably, an uneven surface texture.

More preferably, the bladder is in the form of a bellows, the number used depending on the size of the wound cavity.

Moreover, the bladder comprises two layers, an inner layer and an outer layer, the outer layer being convoluted and having an inlet and a plurality of apertures to introduce media into the wound cavity. In this way, ozone or a similar bacterial growth inhibitor can be introduced to the wound cavity. Alternatively, oxygen could be introduced into the wound cavity for local oxygenation. Similarly, saline solution or liquid disinfectant could be introduced to the wound cavity to inhibit infection. Preferably, the media introduced into the wound cavity is heated to provide normothermic wound surface heating. Alternatively, for a simpler construction, the bladder preferably is of gas permeable material, the fluid inflating the bladder, say for example ozone or oxygen, allowed to permeate into the wound cavity.

Preferably, the bladder is transparent or translucent to allow for visual inspection of the wound without the need for removal of the device. As an additional means of promoting the healing of the wound, the bladder includes light emitting means, for example laser or polychromatic light, or more preferably, electrical stimulation means to stimulate the wound cavity surface.

The invention will be now be further described by way of example, with reference to the accompanying drawings, in which:

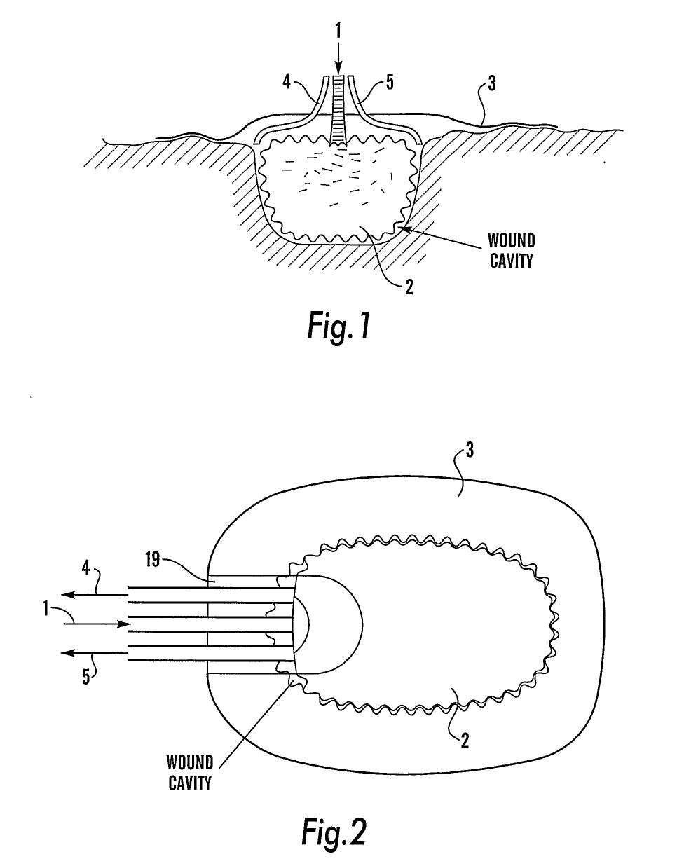

FIG. 1 shows a cross sectional view of one embodiment of a device according to the invention;

FIG. 2 shows the device in FIG. 1 where the inlets to the bladder and vacuum means are coaxially located;

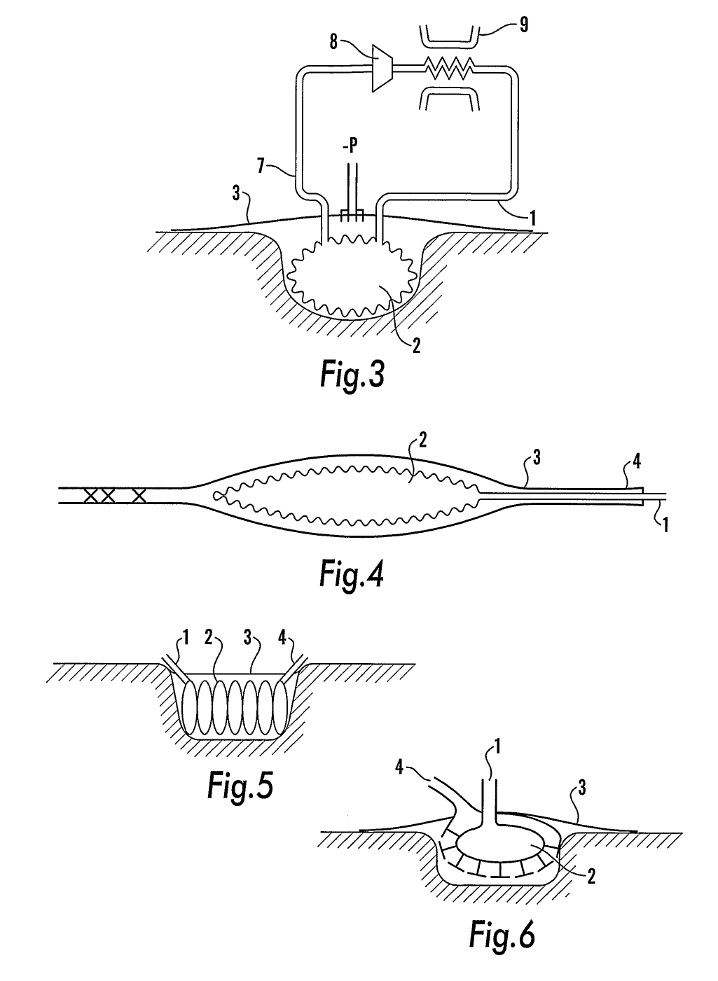

FIG. 3 shows another embodiment of a device providing heating; and

FIGS. 4 to 6 show alternative versions of the bladder for the device.

Referring to FIG. 1, the device consists of a bladder 2, having inlet 1 connected to a fluid source (not shown). The bladder 2 can be of any suitable polymeric material, for example polyurethane, PVC or polyethylene and can also be impregnated with wound healing compounds to promote healing. The bladder 2 is inflated by means of inlet 1 and placed within a wound cavity to generally conform to the cavity shape. A one way valve (not shown) at the inlet 1 maintains the bladder 2 in its inflated state. The bladder 2 can be inflated by any suitable means for example, a hand pump or a compressor. The bladder 2 can be inflated to provide constant pressure against the wound surface or alternatively, with some modifications known in the art can be inflated alternately to provide pulsed pressure. A seal 3, which can be any adhesive film or flexible polymer sheet with adhesive applied to the surface facing the tissue seals the bladder 2 and the wound cavity from atmosphere. The seal 3 overlies the bladder 2 and extends beyond the wound area onto intact tissue. The seal 3 includes one or more inlets/outlets 4, 5 for connection to a vacuum source and/or for the supply of media to be introduced into the wound cavity. The vacuum source can be a vacuum pump or a continuous vacuum provided in hospitals. The media to be introduced into the wound cavity can be saline, water or ozone, oxygen, or any substance promoting wound healing.

In one embodiment, the seal 3 is provided with one tube 4 connected to a vacuum source and supply of media with suitable controls to operate the vacuum and supply as required. Alternatively, the seal 3 can be provided with a plurality of tubes 4, 5 to provide a vacuum 4 and supply of media 5 separately. These two in combination provide a controlled rate of flow of media across the wound surface at a pressure substantially below atmospheric pressure with any wound exudate scavenged along to the vacuum exit tube 4. The tubes 4, 5 can be located coaxially or concentrically in one location on the seal 3 (see FIG. 2) or located in any desired position on the seal 3. The seal 3 can also be provided with a collar 19 to support the tubes 4, 5.

The bladder 2 can simply be evacuated and made smaller when it has to be removed from the wound cavity without causing any damage to the wound surface. Furthermore, during healing of the wound, as the wound cavity shrinks so can the bladder by removal of the inflation fluid. Therefore there is no requirement for changing the wound healing device.

In another preferred embodiment, the bladder inlet and vacuum inlet are operated by the same pump, the pump inflating the bladder insitu within the wound cavity and then switching over to apply a vacuum to the wound cavity. Furthermore, the vacuum can be applied to the bladder to facilitate removal of the bladder from the wound cavity.

As shown in FIG. 3, the bladder 2 is provided with an inlet 1 and an outlet 7 connected to a compressor 8 and heater 9. Thus, the bladder 2 is inflated with heated air to provide heating to the wound surface, the heating promoting perfusion of the area. The air can be replaced by water to provide the same effect.

The bladder 2 can be of transparent or translucent material to allow visual inspection of the wound surface without having to remove the seal. The translucent bladder 2 can also be provided with optical fibres to provide light to the wound surface to stimulate healing. These optical fibres can run co-axially down the inlet 1 tube and terminate at entry to the bladder 2, permitting illumination of the wound cavity. In another embodiment, the walls of the inlet 1 tube itself may act as a light conduit from a light source in the fluid pump, and light evenly dispersed around the wound cavity by the bladder 2 material acting as a diffuser. This is an improvement over current phototherapy treatment methods, where illumination is only possible during change of dressing. With the method described above, illumination can occur continuously if necessary, either at 100% power level or pulsated, and at different wavelengths, under the control of a computer system inside the fluid pump.

Similarly, the wound surface can be subjected to magnetic pulse therapy by using intermittent alternating voltage applied as a coil. Typically, a coil is placed within a sealed cover and manipulated so that the coil axis is at 90° to the wound surface, when energised the coil ensures that the wound surface is placed at an alternating magnetic field of sufficient intensity to promote wound healing.

The bladder 2 can also be impregnated with interdigitated electrodes for direct electrical wound surface stimulation, known to promote healing, or the bladder can be of a conductive material connected electrically.

The wound healing device of the present invention can be used on a range of wounds, the bladders taking any shapes and sizes to accommodate differing wound cavities from shallow to irregular and deep wounds. FIGS. 4 to 6 show some variations of the bladder configurations but any size or shape is possible. FIG. 4 shows the bladder 2 within a shallow wound cavity. FIG. 5 shows the bladder as a series of bellows, the number depending upon the wound size. FIG. 6 shows the bladder having an inner layer and an outer layer.

1-12. (canceled)

13. A wound treatment device comprising:
a. a bladder to be placed over and/or within a wound, the bladder being inflatable to conform to the surface of the wound;
b. sealing means for isolating the bladder from the atmosphere when placed over and/or within the wound;
c. a first passage, the first passage opening adjacent the bladder; and
d. a suction source connected to the first passage, whereby the suction source may apply suction to an area between the bladder and the wound and thereby drain exudates from the area.

14. The wound treatment device of claim 13 wherein the bladder, when inflated, has a surface with folds thereon.

15. The wound treatment device of claim 13 wherein the bladder has a convoluted outer surface.

16. The wound treatment device of claim 13 wherein the bladder has an outer surface with an uneven texture.

17. The wound treatment device of claim 13 wherein the bladder is at least partially defined by a bellows.

18. The wound treatment device of claim 13 further comprising:
a. a second passage, the second passage opening adjacent the bladder; and
d. a fluid rinsing media source connected to the second passage, whereby the fluid rinsing media source may supply fluid rinsing media to an area between the bladder and the wound.

19. The wound treatment device of claim 18 further comprising a heater heating the fluid rinsing media from the fluid rinsing media source, whereby the fluid rinsing media may be heated to promote normothermic wound surface heating.

20. The wound treatment device of claim 13:
a. further comprising a fluid inflating media source connected to the first passage, whereby the fluid inflating media source may supply fluid inflating media to the interior of the bladder;
b. wherein the bladder is formed of material which the fluid inflating media may pass through, whereby fluid inflating media inflating the bladder may permeate into the wound.

21. The wound treatment device of claim 13 wherein the bladder is transparent to allow for visual inspection of the wound.

22. The wound treatment device of claim 13 further comprising a light source within or adjacent the bladder, whereby the light source may photostimulate the wound surface.

23. The wound treatment device of claim 13 further comprising an electrical terminal within or adjacent the bladder, whereby the terminal may electrically stimulate the wound surface.

24. The wound treatment device of claim 13 further comprising a fluid inflating media source connected to the first passage, the fluid inflating media source being actuatable to alternately supply and withdraw fluid inflating media to and from the interior of the bladder.

25. The wound treatment device of claim 13 wherein the sealing means includes a skirt extending from the bladder.

26. The wound treatment device of claim 25 wherein at least a portion of the skirt bears adhesive.

27. A wound treatment device comprising:
a. a seal, the seal being defined by a sheet of material having opposing sides;
b. an inflatable bladder extending from at least one of the sides of the seal, whereby the seal defines a skirt extending outwardly from at least a portion of the bladder;
c. an inflating fluid media source in fluid communication with the bladder, whereby the bladder may be inflated with inflating fluid media.

28. The wound treatment device of claim 27 wherein at least one of the opposing sides of the seal bears adhesive.

29. The wound treatment device of claim 27 further comprising a rinsing fluid media source providing rinsing fluid media to at least one opening situated adjacent the bladder, whereby the area adjacent the bladder may be rinsed with rinsing fluid media.

30. The wound treatment device of claim 27 further comprising a suction source having at least one opening situated adjacent the bladder, whereby the suction source may apply suction to an area adjacent the bladder.

31. A wound treatment device comprising:
a. a seal, the seal being defined by a sheet of material having opposing sides bounded by a seal perimeter;
b. an inflatable bladder spaced inwardly from the seal perimeter, whereby the seal defines a skirt extending outwardly from the bladder;
c. an inflating fluid media source in fluid communication with the bladder, whereby the bladder may be inflated with inflating fluid media; and
d. a fluid communication passage:
(1) isolated from the interior of the bladder, and
(2) opening adjacent the bladder,
e. a fluid pressure source in communication with the fluid communication passage, whereby the fluid pressure source may supply fluid to, and/or withdraw fluid from, the fluid communication passage.

32. The wound treatment device of claim 31 wherein at least one of the opposing sides of the seal bears adhesive.
